(19) United States
(12) Patent Application Publication
(10) Pub. No.: US 2024/0383824 A1
(43) Pub. Date: Nov. 21, 2024

(54) INTEGRATED PROCESS FOR CONVERTING CARBON OXIDE TO OLEFINS

(71) Applicant: UOP LLC, Des Plaines, IL (US)

(72) Inventors: John J. Senetar, Naperville, IL (US); Ian G. Horn, Streamwood, IL (US); Andrew B. Chin, Wilmette, IL (US)

(21) Appl. No.: 18/653,874

(22) Filed: May 2, 2024

Related U.S. Application Data
(60) Provisional application No. 63/466,704, filed on May 15, 2023.

(51) Int. Cl. C07C 1/24; C07C 7/00; C07C 29/151; C07C 29/74

(52) U.S. Cl. CPC C07C 1/24; C07C 7/005; C07C 29/151; C07C 29/74

(57) ABSTRACT

An integrated process for producing light olefins by integrating methanol purification with olefin production from methanol. The methanol fed to the MTO process may be crude methanol or purified methanol with the heavy oxygenates removed and treated with heavy oxygenates in the MTO effluent. By eliminating or reducing the need for purification of methanol by the methanol supplier, the cost will be reduced for methanol conversion to olefins or fuels.

### FIELD

The field is related to an integrated process for producing light olefins from carbon oxide. The field may particularly relate to integrating a methanol synthesis process with an oxygenate conversion process.

### BACKGROUND

Olefins have been traditionally produced from petroleum feedstock by catalytic or steam cracking processes. These cracking processes, especially steam cracking, produce light olefins such as ethylene and propylene from a variety of hydrocarbon feedstock. Ethylene and propylene are important commodity petrochemicals useful in a variety of processes for making plastics and other chemical compounds.

The petrochemical industry has known for some time that oxygenates, especially alcohols, are convertible into light olefins. For example, methanol, the preferred alcohol for light olefin production, may be converted to primarily ethylene and propylene in the presence of a molecular sieve catalyst. This process is referred to as a methanol-to-olefin (MTO) reaction process, which occurs in an MTO reaction system. The highly efficient MTO process may convert oxygenates to light olefins which had been typically used for plastics production. Light olefins produced from the MTO process are concentrated in ethylene and propylene but include C4-C6 olefins.

Methanol is typically synthesized from the catalytic reaction of syngas in a methanol reactor in the presence of catalyst. Syngas is defined as a gas comprising primarily carbon monoxide (CO), hydrogen (H₂) and preferably carbon dioxide (CO₂). Other components may also be present. Syngas production processes are well known, and include conventional steam reforming, autothermal reforming, dry reforming or a combination thereof.

Methanol synthesis is known to generate a number of byproduct impurities including methane, dimethyl ether, methyl formate, higher alcohols, ketones (e.g., acetone, methyl ethyl ketone). The amounts and types of these impurities depend on the feedstock and the method employed for methanol synthesis. Effective use of the product methanol generally requires capital and energy-intensive separation steps to remove impurities both more volatile than methanol and less volatile than methanol. These separations negatively impact the overall economics for methanol production. Downstream use of use of methanol for generation of olefins, gasoline, jet fuel and distillate generate the same byproduct impurities that occur in methanol synthesis. In addition, byproducts such as acetaldehyde are also present. As in methanol synthesis, these impurities require considerable capital and energy costs for removal.

An effective means is desired for integrating the purification steps for methanol synthesis and for product purification steps for the downstream methanol conversion.

### BRIEF SUMMARY

We have formulated an integrated process for producing light olefins by integrating methanol purification with olefin production from methanol. The methanol fed to an MTO process may be crude methanol or purified methanol with the heavy oxygenates removed and treated with heavy oxygenates in the MTO effluent. By eliminating or reducing the need for purification of methanol by the methanol supplier, the cost will be reduced for methanol conversion to olefins or fuels.

### BRIEF DESCRIPTION OF THE DRAWINGS

FIG. 1 is a schematic drawing of a methanol synthesis and oxygenate conversion process in accordance with the present disclosure.

FIG. 2 is a schematic drawing of a MTO recovery process in accordance with an exemplary embodiment of an integrated process for producing light olefins of the present disclosure.

FIG. 3 is a schematic drawing of an alternative methanol synthesis and oxygenate conversion process in accordance with the present disclosure.

FIG. 4 is a schematic drawing of another alternative methanol synthesis and oxygenate conversion process in accordance with the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripping columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5”, “T10”, “T90” or “T95” means the temperature at which 5 mass percent, 10 mass percent, 90 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “diesel” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method or a T90 between 280° C. (536° F.) and about 340° C. (644° F.) using ASTM D-86. The term “green diesel” means diesel comprising hydrocarbons not sourced from fossil fuels.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” means jet fuel comprising hydrocarbons not sourced from fossil fuels.

As used herein, the term “a component-rich stream” means that the rich stream coming out of a vessel has a greater concentration of the component than the feed to the vessel and preferably than all other streams withdrawn from the vessel.

As used herein, the term “a component-lean stream” means that the lean stream coming out of a vessel has a smaller concentration of the component than the feed to the vessel and preferably than all other streams withdrawn from the vessel.

As used herein, the term “ppmw” designates parts per million by weight.

As used herein, the term “C₂₊ oxygenate” means a molecule, typically a hydrocarbon, comprising an oxygen atom and at least two carbon atoms.

### DETAILED DESCRIPTION

An integrated process and apparatus for producing methanol from carbon oxide and converting methanol into light olefins and perhaps to fuels is disclosed. In one embodiment, crude methanol is fed directly to an MTO unit. This embodiment takes advantage of reacting the DME and other oxygenates in the crude methanol to produce additional olefins. Only a portion of the heavy oxygenates in the methanol feed will react in the MTO reactor; however, the product recovery section for the MTO reactor effluent can be used to recover the unreacted oxygenates. The major light impurity in crude methanol is carbon dioxide. The additional carbon dioxide in the MTO reactor effluent increases the duty for carbon dioxide removal downstream in the caustic scrubber. To address the additional carbon dioxide, bulk carbon dioxide removal is employed upstream of the caustic scrubber to remove the carbon dioxide to lessen the caustic consumption. An added benefit is that the bulk carbon dioxide removal provides additional carbon dioxide that can be utilized in other means such as producing syngas that will be provide feed to the methanol conversion unit.

In a second embodiment, crude methanol fractionation columns are employed in the MTO unit. The energy requirements for operating these columns can be supplied by waste energy from the MTO unit. Also, the purified methanol product can be taken overhead as a vapor and sent directly to the MTO reactor as feed instead of conventionally condensing it at the conclusion of the purification process and subsequently vaporizing it for the MTO reactor. In the conventional MTO process, liquid methanol is vaporized before it is fed to the MTO unit, thereby consuming energy to vaporize the methanol. Additionally, the specifications for the methanol distillation can be relaxed and thereby reducing the related capital and energy consumption. Water containing heavy oxygenates can be processed along with the water condensed from the MTO reactor effluent.

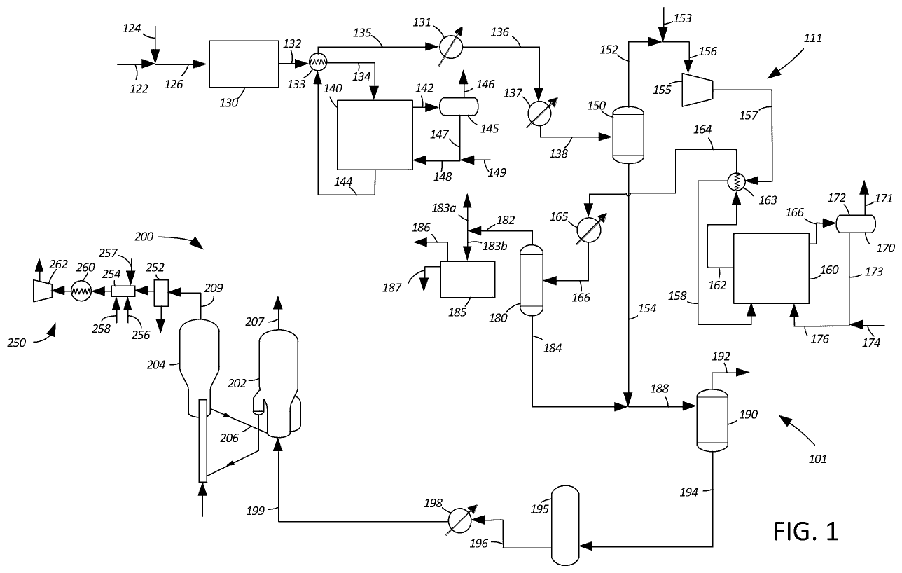

Turning to FIG. 1 of an integrated process and apparatus 101 for producing light olefins comprises a methanol synthesis section 111 and an oxygenate conversion unit 200. As shown in FIG. 1, a syngas stream in line 122 and a hydrogen gas stream in line 124 are passed to the methanol synthesis section 111. Syngas is defined as a gas comprising primarily carbon monoxide (CO), carbon dioxide (CO₂) and hydrogen (H₂). Optionally, syngas may also include methane (CH₄), and small amounts of ethane and propane. Conventional processes for converting carbon molecules to syngas include steam reforming, partial oxidation, autothermal reforming, and combinations of these processes. In accordance with an embodiment of the present disclosure, the syngas stream in line 122 may be taken from any suitable source. In accordance with another embodiment of the present disclosure, the hydrogen gas stream in line 124 may be taken from any suitable source. In an exemplary embodiment, the hydrogen gas stream in line 124 is taken from a pressure swing adsorption (PSA) unit. In an alternate embodiment, the hydrogen gas stream in line 124 may be taken from a water electrolysis unit.

In accordance with an exemplary embodiment, of the present disclosure, the methanol synthesis section 111 comprises a first methanol converter 140 and a second methanol converter 160. The syngas stream in line 122 and the hydrogen gas stream in line 124 are passed to the first methanol converter 140 of the methanol synthesis section 111. In an embodiment, the syngas stream in line 122 and the hydrogen gas stream in line 124 may be combined to provide a combined feed stream 126 which is passed to the first methanol converter 140. However, the syngas stream in line 122 and the hydrogen gas stream in line 124 may be passed separately to the first methanol converter 140. The combined feed stream 126 may be passed to a syngas pressure booster compressor 130 to compress the syngas to a particular pressure to provide a compressed syngas stream in line 132 before passing it to the first methanol converter 140. In an exemplary embodiment, the syngas may be compressed to a pressure from about 6890 kPa (1000 psia) to about 8970 kPa (1300 psia) in the syngas pressure booster compressor 130. The syngas stream may be heated before passing it to the first methanol converter 140. The compressed syngas stream in line 132 may be heat exchanged in a heat exchanger 133 with a first reactor effluent stream in line 144 to provide a heated syngas stream in line 134. The heated syngas stream in line 134 is passed to the first methanol converter 140.

In the first methanol converter 140 of the methanol synthesis section 111, the syngas is converted to a methanol composition. The methanol synthesis process is accomplished in the presence of a methanol synthesis catalyst. In an exemplary embodiment, the syngas stream in line 122 to the methanol synthesis section 111 has a molar ratio of carbon dioxide to carbon monoxide of between 1:2 and 1:4 and a molar ratio of hydrogen to carbon oxides (CO+CO2) in the range of from about 3:2 to about 3:1.

A suitable methanol synthesis catalyst may be a copper on a zinc oxide and alumina support. Synthesis conditions of the first methanol converter 140 of the methanol synthesis section 111 may include a temperature of about 200 to about 300° C. and a pressure of about 3.5 to about 10 MPa. Reaction equilibrium typically requires methanol separation and recycle of unreacted reagents to the synthesis reaction to obtain sufficient conversion.

In accordance with an exemplary embodiment, the first methanol converter 140 may operate at a temperature of about 204° C. (400° F.) to about 290° C. (550° F.). In accordance with another exemplary embodiment, the first methanol converter 140 may operate at a pressure from about 6890 kPa (1000 psia) to about 8970 kPa (1300 psia).

The methanol synthesis reaction is highly exothermic. A boiler feed water (BFW) stream in line 148 is passed to the first methanol converter 140 to generate a steam stream in line 142, which is withdrawn from the first methanol converter 140. The generation of steam absorbs the exotherm in the methanol synthesis reaction. The steam stream in line 142 is passed to a steam separator 145 to separate steam in line 146 from a water stream in line 147. The water stream in line 147 is supplemented with a recycled BFW in line 149 to provide the BFW in line 148 for the first methanol converter 140.

In the first methanol converter 140, the syngas is converted to a methanol composition in a first reactor effluent comprising methanol in line 144. The methanol stream in the first reactor effluent in line 144 may include methanol, dimethyl ether, ethanol or combinations thereof. The first reactor effluent stream in line 144 is heat exchanged in the heat exchanger 133 with the compressed syngas stream in line 132. A heat exchanged first reactor effluent stream in line 135 may be cooled in a first cooler 131 to provide a cooled first reactor effluent stream in line 136. The cooled first reactor effluent stream in line 136 may be further cooled in a second cooler 137 to provide a further cooled first reactor effluent stream in line 138. The further cooled first reactor effluent stream in line 138 is separated in a first gas-liquid separator 150 to provide a first vapor stream in line 152 and a first liquid stream in line 154. The first vapor stream in line 152 and the first liquid stream in line 154 may be further processed to recover methanol.

The first vapor stream in line 152 comprises carbon dioxide that has not yet converted to methanol. The first vapor stream in line 152 may be compressed in a first compressor 155. In an embodiment, the first vapor stream in line 152 may be combined with a make-up hydrogen stream in line 153 to provide a combined first vapor stream in line 156. The combined first vapor stream in line 156 is compressed in the first compressor 155 to provide a compressed first vapor stream in line 157 at a pressure from about 6890 kPa (1000 psia) to about 8970 kPa (1300 psia). In an embodiment, the make-up hydrogen stream in line 153 may be taken from any suitable sources. In accordance with the present disclosure, the make-up hydrogen stream in line 153 may be taken from one or more units of the process and apparatus 101.

The compressed first vapor stream in line 157 is heated by heat exchange with a second reactor effluent stream in line 162 in the heat exchanger 163 to provide a heated first vapor stream in line 158 which is passed to the second methanol converter 160. In the second methanol converter 160 of the methanol synthesis section 111, the unconverted carbon dioxide in the syngas is converted to a methanol composition. The methanol synthesis process is accomplished in the presence of a methanol synthesis catalyst. A suitable methanol synthesis catalyst may be a copper on a zinc oxide and alumina support. Synthesis conditions of the second methanol converter 160 of the methanol synthesis section 111 may include a temperature of about 200° C. (390° F.) to about 300° C. (570° F.) and a pressure of about 3.5 MPa(g) (510 psig) to about 10 MPa(g) (1450 psig). Reaction equilibrium typically requires methanol separation and recycle of unreacted reagents to the synthesis reaction.

A boiler feed water (BFW) stream in line 176 is passed to the second methanol converter 160 to generate a steam stream in line 166 withdrawn from the second methanol converter 160 to absorb the exotherm. The steam stream in line 166 is passed to a steam separator 172 to separate steam in line 171 from a water stream in line 173. The water stream in line 173 is supplemented with a recycled BFW in line 174 to provide the BFW in line 176 for the second methanol converter 160.

In the second methanol converter 160, the first reactor effluent stream is converted to a methanol composition to provide a second reactor effluent stream comprising methanol in line 162. The methanol stream in the second reactor effluent stream in line 162 may include methanol, dimethyl ether, ethanol or combinations thereof. The second reactor effluent stream in line 162 is cooled by heat exchange in the heat exchanger 163 with the compressed first vapor stream in line 157. A heat exchanged second reactor effluent stream in line 164 may be cooled in a cooler 165 to provide a cooled and condensed second reactor effluent stream in line 166. The cooled second reactor effluent stream in line 166 is separated in a second gas-liquid separator 180 to provide a second vapor stream in line 182 and a second liquid stream in line 184. The second vapor stream in line 182 and the second liquid stream in line 184 may be further processed to recover methanol.

In accordance an exemplary embodiment, the second methanol converter 160 is operated at a temperature of about 204° C. (400° F.) to about 290° C. (550° F.). In accordance with another exemplary embodiment, the second methanol converter 160 is operated at a pressure from about 6890 kPa (1000 psia) to about 8970 kPa (1300 psia).

In accordance with the present disclosure, the second vapor stream in line 182 is passed to a PSA unit 185 to separate hydrogen from the second vapor stream in line 182. In an exemplary embodiment, the second vapor stream in line 182 may be separated into a recycle stream in line 183a and a PSA feed stream in line 183b. In another exemplary embodiment, the recycle stream in line 183a may be passed to the first compressor 155 as the make-up hydrogen stream 153. In an embodiment, the make-up hydrogen stream in line 153 to the first compressor 155 comprises the recycle stream in line 183a.

The PSA feed stream in line 183b is processed in the PSA unit 185. Typically, a PSA unit includes a series of multiple adsorber beds containing one or a combination of multiple adsorbents suitable for adsorbing the particular components to be adsorbed therein. These adsorbents include, but are not limited to, activated alumina, silica gel, activated carbon, zeolite molecular sieve type materials, or any combination thereof. The adsorbents are organized in any sequence as required by the adsorption process to adsorb impurities or components. In the PSA unit 185, PSA feed gas flows over the adsorbent and the more readily adsorbable impurities are adsorbed during the adsorption step while hydrogen flows through. Pressure swing enables adsorbed impurities on the adsorbent to desorb into line 186. The purified hydrogen gas leaves the adsorber bed in the PSA product gas stream 187 that is lean in impurities.

In the PSA unit 185, hydrogen present in the PSA feed stream in line 183b is separated into a hydrogen rich stream in line 187. As shown, from the PSA unit 185, a purge stream in line 186 is separated from the hydrogen rich stream in line 187. The purge stream in line 186 may be used as fuel. In an exemplary embodiment, the hydrogen rich stream in line 187 may be passed to the syngas pressure booster compressor 130 as the hydrogen stream in line 124. In an embodiment, the hydrogen stream in line 124 to the syngas pressure booster compressor 130 comprises the hydrogen rich stream in line 187.

Turning back to the second gas-liquid separator 180, the second liquid stream in line 184 is withdrawn from the bottom of the second gas-liquid separator 180 and passed to a third gas-liquid separator 190. The first liquid stream in line 154 may also be passed to the second gas-liquid separator 180. In an exemplary embodiment, the second liquid stream in line 184 may be combined with the first liquid stream in line 154 to provide a combined liquid stream in line 188 which is passed to the third gas-liquid separator 190. In the third gas-liquid separator 190, the first liquid stream in line 154 and the second liquid stream in line 184 are separated into a third vapor stream in line 192 and a third liquid stream in line 194. The third liquid stream in line 194 comprises crude methanol. Alternately, the third liquid stream in line 194 may be a crude methanol stream. The crude methanol stream may comprise at least 100 ppmw of carbon oxide and/or at least 100 ppmw C₂₊ oxygenates.

The crude methanol comprises methanol, light ends, and heavier alcohols. As used and described herein, the term “crude methanol” or “crude oxygenate feedstock” may comprise methanol, ethanol, water, light ends, and fusel oil. The light ends may include ethers such as dimethyl ether (DME), ketones such as acetone, aldehydes such as acetaldehyde, and dissolved gases such as hydrogen, methane, carbon oxides, and nitrogen. The fusel oil in the crude methanol typically includes higher molecular weight alcohols and is generally burned as a fuel in the methanol plant. The crude methanol comprising the fusel oil can be passed to the oxygenate conversion unit for the additional production of light olefins. In accordance with the present disclosure, the crude methanol may be passed directly to the oxygenate conversion unit or the MTO unit as feed.

In accordance with an exemplary embodiment of the present disclosure, the crude methanol may have a composition comprising carbon monoxide in a concentration from about 0 to about 1 wt %, carbon dioxide in a concentration from about 0.05 wt % to about 2 wt %, methane in a concentration from about from about 0.001 wt % to about 2 wt %, hydrogen in a concentration from about 0.05 wt % to about 2 wt %, oxygen in a concentration from about 0 to about 1 wt %, water in a concentration from about 5 wt % to about 18 wt %, nitrogen in a concentration from about 0 to about 1 wt %, methanol in a concentration from about 75 wt % to about 90 wt %, and heavy alcohols having at least 2 carbon atoms in a concentration from about 0.05 to about 4 wt %.

The third liquid stream in line 194 may be passed to a crude methanol hold-up tank 195. A crude methanol stream in line 196 is withdrawn from the crude methanol hold-up tank 195. In accordance with the present disclosure, the crude methanol stream in line 196 may be passed to the oxygenate conversion unit 200 as shown in FIG. 1. The crude methanol hold-up tank 195 is optional.

Conventionally, the crude methanol stream in line 196 is purified of the light gases and heavy oxygenates before it is charged to a MTO reactor 202 in an oxygenate conversion unit 200. In accordance with the embodiment of FIG. 1, the crude methanol stream comprising at least 100 ppmw of carbon oxide and/or at least 100 ppmw C₂₊ oxygenates is passed directly to the MTO reactor 202 after it is preheated and vaporized in heat exchanger 198. Heat exchanger 198 may include a series of heat exchangers that use waste heat from the process to preheat and vaporize the crude methanol stream in line 196.

A methanol charge stream in line 199 is fed to the MTO reactor 202 that reacts an oxygenate such as methanol or dimethyl ether (DME) with a catalyst. In an embodiment, the methanol charge stream in line 199 comprises a superheated crude methanol stream. In the MTO reactor 202, the methanol charge stream is contacted with an MTO catalyst at MTO reaction conditions to convert methanol and other oxygenates to olefins and water. The crude methanol stream in line 198 may include methanol, dimethyl ether, ethanol or combinations thereof. The MTO reactor 202 may fluidize catalyst at fast fluidized conditions. The MTO catalysts may be a silicoaluminophosphate (SAPO) catalyst. SAPO catalysts and their formulation are generally taught in U.S. Pat. No. 4,499,327A, U.S. Pat. No. 10,358,394 and U.S. Pat. No. 10,384,986. The MTO reaction conditions include contact with a SAPO catalyst at a pressure between about 0.1 MPa(a) and about 0.7 MPa(a) (about 15 psia to about 100 psia). The MTO reaction temperature should be between about 400° C. (750° F.) to about 525° C. (980° F.). A weight hourly space velocity (“WHSV”) in the MTO reactor 202 is in the range of about 1 to about 15 hr⁻¹. The MTO catalyst is separated from the product olefin stream after the MTO reaction.

In the MTO process, catalyst particles are repeatedly circulated between the MTO reactor 202 and the MTO regenerator unit 204. During regeneration, coke deposited on the catalyst particles during reaction in the reaction zone is removed at elevated temperatures by oxidation in the regenerator unit 204. The removal of coke deposits restores the activity of the catalyst particles to the point where they can be reused in the MTO reactor 202. The regenerated catalyst is discharged from the regenerator unit 204 in line 206 and recycled to the MTO reactor 202. A MTO effluent stream of light olefins comprising ethylene and propylene and other olefins along with water and oxygenates are discharged from the MTO reactor 202 in an effluent line 207.

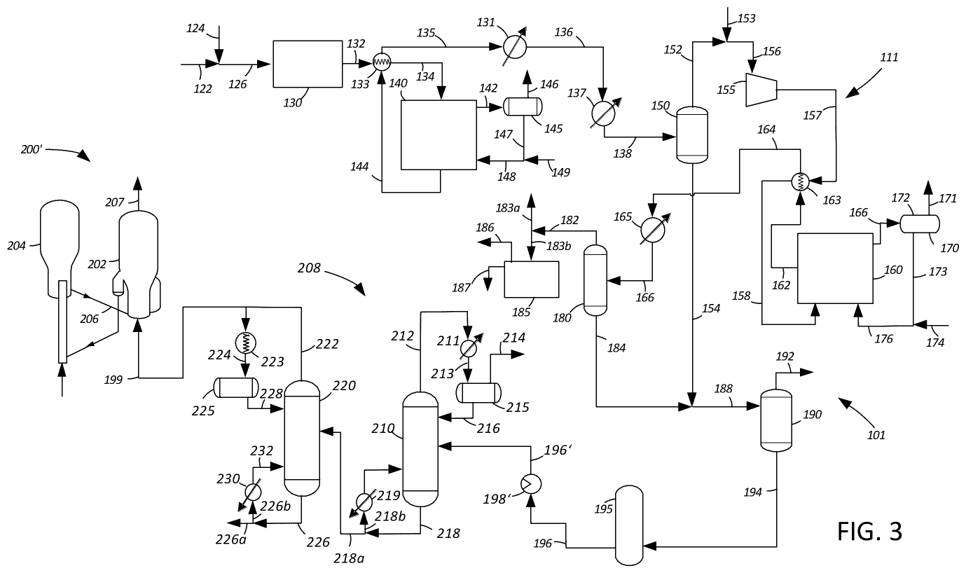

The oxygenate conversion unit 200 in FIG. 1 may comprise a flue gas recovery section 250 for recovery of heat from hot flue gas in line 209 discharged from the catalyst regenerator 204. The flue gas is very hot and can be used to recover heat from the catalyst regeneration process. The flue gas in line 209 will contain catalyst fines which may be removed by a fines removal unit 252 comprising a third stage separator or a filter. Flue gas with fines removed down to a very low concentration may comprise carbon monoxide that can be combusted to carbon dioxide in a combustor 254. A fuel gas stream in line 256 and optionally a heavy oxygenates stream in line 258 may be provided to the combustor 254 to further increase the temperature of the flue gas. Additional oxygen may be added in line 257 as either air or another oxygen containing stream. Extremely hot flue gas may be passed to a steam generator 260 to generate high pressure steam by heat exchange between water and the flue gas stream. Additionally, kinetic energy may be recovered from the flue gas stream by passing it through a turbine 262 to provide work. The flue gas recovery process can be used in the embodiment of FIG. 3 as well.

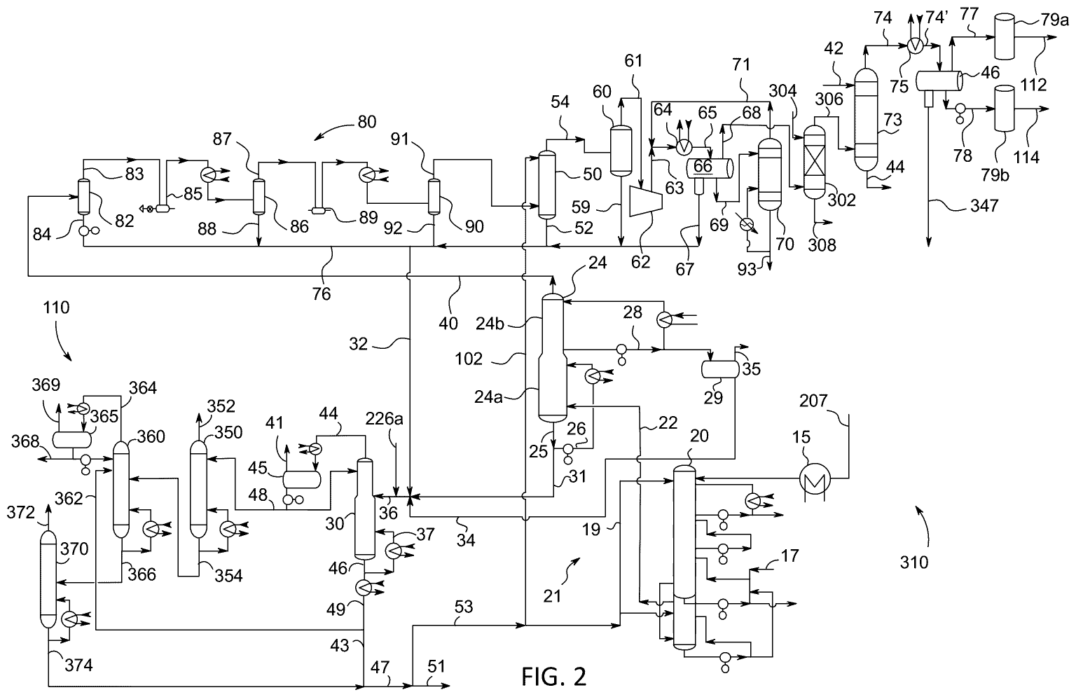

Line 207 transports MTO products in a MTO effluent stream to an MTO product unit 310 illustrated in FIG. 2. The MTO product unit 310 may separate light olefins from oxygenates for further valorization. The MTO product unit 310 comprises a MTO separation section 21, oxygenate separation section 110, and a concentration section 80. The MTO separation section 21 comprises a water stripper column 30, a quench column 20 and a product separator column 24. The oxygenate separation section 110 comprises a DME stripper column 350, an extractive distillation column 360, and a methanol stripper column 370.

As shown in FIG. 2, the hot vaporous MTO effluent stream in line 207 may be preliminarily cooled in a reactor effluent heat exchanger 15 to recover heat before it is passed to a quench column 20. In an aspect, the hot vaporous MTO effluent stream in line 207 may produce two levels of steam in the reactor effluent heat exchanger 15 to cool it before passing it to the quench column 20. In the quench column 20, the vaporous reactor effluent is desuperheated, neutralized of organic acids and clarified of catalyst fines by direct contact with a water stream supplied in line 19 which may be taken from a water rich stream in line 47. In addition, circulated water streams in the quench tower system are employed in multiple stages to enhance catalyst fines recovery. Additional sections in quench column 20 may be provided for caustic injection to remove organic acids such as acetic acid and entrained caustic from a caustic contacting section. A quenched olefin stream in line 22 is discharged from the quench column 20 and fed to a product separator column 24 in the separation section 21.

The product separator column 24 comprises two sections a first, or lower, section 24a and a second, or upper section 24b for separating the reactor effluent stream into a product olefin stream in an overhead line 40, an intermediate liquid stream in an intermediate line 28 and a water stream in a bottoms line 25. The water stream in the bottoms line 25 may be separated into a product water stream in bottoms line 31 and a recycled product water stream in line 26. A first, or lower, section 24a receives the quenched reactor effluent stream in line 22. In the lower section, a portion of the heat is removed from the quenched reactor effluent stream while partially condensing the water in the quenched reactor effluent stream to generate the product water stream in bottoms line 25 comprising a portion of the oxygenate byproducts in the quenched reactor effluent stream in line 22. A portion of the product water stream is cooled and pumped in line 26 around to the top of the first section 24a of the product separator column 24 in the recycled product water stream to cool the quenched reactor effluent stream in line 22.

In accordance with an embodiment of the present disclosure, a net product water stream in line 31 is taken from the product water stream in the bottoms line 25 and passed to a water stripper column 30. A water return stream comprising oxygenate byproducts from the concentration section 80 in return line 32 can also be passed to the water stripper column 30. A coalescer bottoms line 34 may transport an aqueous stream in line 34 to the water stripper column 30 from a coalescer 29. Lastly, an aqueous oxygenates stream in a net oxygenates bottoms line 226a from the embodiment of FIG. 3 may also be delivered to the water stripper column 30. The water stripper column 30 may be in downstream communication with the product separator column 24, the coalescer 29 and the concentration section 80. A water stripper feed in line 36 may deliver the streams in lines 26, 32, 34 and 226a to the water stripper column 30.

A vapor stream from the first section 24a of the product separator column 24 is passed to the second, or upper, section 24b of the product separator. An intermediate stream in line 28 comprising hydrocarbons, oxygenate byproducts, and water in liquid phase is withdrawn at a bottom of the upper section 24b. A portion of the intermediate stream in line 28 is cooled and passed to the top of the second section 24b of the product separator column 24. The remainder of the intermediate stream in line 28 is passed to a coalescer 29 to separate a hydrocarbon overhead stream from an aqueous stream in line 34 which is fed back to the product water stream in line 26 and pumped to the water stripper column 30. An overhead product olefin stream comprising olefins from the second section 24b of the product separator column 24 in line 40 is delivered to the concentration section 80. In accordance with an exemplary embodiment, the product water stream in line 26, the aqueous stream in line 34, and the water return stream in line 32 are combined to provide a combined product water stream in line 36. The combined product water stream in line 36 is passed to the water stripper column 30. Also, the product water stream in line 26, the aqueous stream in line 34, and the water return stream in line 32 can be passed separately to the water stripper column 30. A net oxygenates bottoms line 226a may deliver an aqueous oxygenate stream to the combined product water stream 36 for processing from the alternative embodiment of FIG. 3.

The combined product water stream in line 36 includes dilute hydrocarbon oxygenates such as dimethyl ether (DME), methanol, acetaldehyde, acetone, methyl ethyl ketone (MEK), and other heavy oxygenates comprising two or more carbon atoms. The water stripper column 30 separates or strips the oxygenates into a methanol and oxygenate rich stream in an overhead line 44 rich in both methanol and at least another oxygenate including heavy oxygenates and a water rich stream in a bottoms line 46. In one embodiment the water stripper column 30 temperature may be about 115° C. (239° F.) to about 180° C. (356° F.) at the bottom of the water stripper column and the pressure may be about 75 kPa(g) (11 psig) to about 1035 kPa(g) (150 psig) at the overhead of the water stripper column 30.

The combined product water stream in line 36 comprising the product water stream in the line 26 includes dilute hydrocarbon oxygenates such as DME, methanol, acetaldehyde, acetone and MEK. The water stripper column 30 separates or strips the oxygenates into a methanol and oxygenate rich stream in an overhead line 44 rich in both methanol and at least another oxygenate and heavy oxygenates and a water rich stream in a bottoms line 46.

A portion of the water rich stream in the bottoms line 46 is reboiled and returned to the water stripping column 30 in a reboil line 37. The remainder of the water rich stream in line 46 is cooled in a cooler to provide a net water rich stream in line 49. The net water rich stream in the line 49 can be divided into a solvent stream provided in line 362 to an extractive distillation column 360 and a bottoms water rich recycle stream in the remaining bottoms line 43. The water rich recycle stream in line 43 is combined with stream in line 374 to provide a water stream 47. The water stream in line 47 can be fed to the quench column 20 in line 19 and to the oxygenate absorber in line 102.

Uncondensed light hydrocarbons can be purged via a first receiver vapor stream in line 41 of the water stripper column 30 while a methanol and oxygenate rich stream can be removed in a net overhead liquid line 48 and comprises methanol, DME, acetaldehyde, acetone and MEK and heavy oxygenates. A portion of the hydrocarbon lean, methanol and oxygenate rich stream can be returned to the water stripper column 30 as reflux. In one embodiment the water stripper column 30 temperature may be about 115° C. (239° F.) to about 150° C. (302° F.) at the bottom of the water stripper column and the pressure may be about 75 kPa gauge (11 psig) to about 345 kPa (50 psig) at the top of the water stripper column.

The methanol and oxygenate rich stream in line 48 may be fed to the extractive distillation column 360 to separate methanol from at least one other oxygenate. However, the methanol and oxygenate rich stream in line 48 comprises DME which easily separates from methanol. Hence, the methanol and oxygenate rich stream in line 48 may be fed to a DME stripper column 350 to easily remove the DME. The DME stripper column 350 may be in downstream communication with the water stripper column 30. The DME stripper column 350 may separate or strip DME into a DME rich stream in an overhead line 352 and provide a DME lean, methanol and oxygenate rich stream in a bottoms line 354. The DME rich stream in the overhead line 352 may be recycled to the oxygenate conversion section 200 as MTO feed. A portion of the DME lean, methanol and oxygenate rich stream from the bottoms line 354 may be reboiled and recycled to the DME stripper column 350. The net, DME lean, methanol and oxygenate rich stream in bottoms line 354 may be fed to the extractive distillation column 360. The extractive distillation column 360 may be in downstream communication with the water stripper column 30 and upstream of any communication with the product separator column 24 to assure that no inert oxygenates build up in the compression section without an avenue for return to the water stripper column 30. Additionally, in an embodiment, the extractive distillation column 360 may be in downstream communication with the DME stripper column 350.

In one embodiment the DME stripper column 350 temperature may be about 85° C. (185° F.) to about 120° C. (248° F.) at the bottom of the DME striper column and the pressure may be about 75 kPa(g) (11 psig) to about 414 kPa(g) (60 psig) at the top of the column. The DME stripper column 350 may utilize an overhead condenser and receiver separator in addition to or instead of the overhead condenser and receiver 45 for the water stripper column 30 to remove a light hydrocarbon purge.

The DME lean, methanol and oxygenate rich stream in the DME stripper net bottoms line 354 may be fed to an extractive distillation column 360 to separate methanol from at least one other hydrocarbon oxygenate and preferably all other hydrocarbon oxygenates. A solvent stream of water in line 362 may also be fed to the extractive distillation column 360 at a location, such as at the top quarter of the column, above a location, such as the middle quarter of the column, at which the DME lean, methanol and oxygenate rich stream is fed to the column. The solvent stream may be provided in line 362 which may be taken from the water rich stream in the water stripper bottoms line 49.

The flow rate of the solvent stream of water to the extractive distillation column 360 should be about 1.5 to about 3 times that of the flow rate of hydrocarbon oxygenates to the extractive distillation column 360 in the DME lean, methanol and oxygenate rich stream in the DME stripper bottoms line 354 and about 1 to about 3 times the flow rate of the entire DME, lean, methanol and oxygenate rich stream in the bottoms line 354 which will also comprise substantial water.

The extractive distillation column 360 produces an oxygenate rich stream in an overhead line 364 comprising the at least one other hydrocarbon oxygenate, such as acetone, acetaldehyde, MEK and DME, heavy oxygenates, and a methanol and water rich stream in a bottoms line 366. A portion of the methanol and water rich stream in the bottoms line 366 may be reboiled and returned to the extractive distillation column 360. The oxygenate rich stream in the overhead line 364 may be cooled and partially condensed and fed to a receiver separator 365. Uncondensed light hydrocarbons can be purged from a second receiver vapor stream in line 369 while a methanol lean oxygenate rich stream can be removed in a receiver liquid line 368 and comprises heavy oxygenates, DME, acetaldehyde, acetone and MEK. Heavy oxygenates fed to the MTO reactor 202 in the crude methanol stream will be concentrated into the methanol lean oxygenate rich stream in line 368. The methanol lean oxygenate rich stream in line 368 may be fed to the combustor 254 of FIG. 1 in line 258. A portion of the methanol lean oxygenate rich stream can be returned to the extractive distillation column 360 as reflux at a location above the location at which the solvent stream is added to the extractive distillation column 360 in line 362.

The light hydrocarbon purge(s) in the first receiver vapor stream in line 41 and the second receiver vapor stream in line 369 may be used as fuel gas. In an embodiment a portion or all of these streams may go to the combustor 254 in FIG. 1 as part of the fuel gas stream in line 256. The methanol lean oxygenate rich stream in line 368 may be combusted to generate heat or steam that may be used elsewhere in the process by sending it to the combustor 254 in FIG. 1 as part of the heavy oxygenates stream in line 258. Alternatively, the oxygenates in line 368 may be recovered for further valorization or to provide feed to the MTO reactor 202. Heavy oxygenates that are not removed from the crude methanol stream in line 199 are recovered here in the methanol lean oxygenate rich stream in line 368.

At least 99 wt %, and preferably at least 99.5 wt %, of the hydrocarbon oxygenates other than methanol fed to the extractive distillation column 360 may be recovered in the methanol lean, oxygenate rich stream in the liquid line 368 of the receiver separator 365. At least 90 wt %, and preferably at least 95 wt %, of the methanol may be recovered in the methanol and water rich stream in the net bottoms line 366.

The extractive distillation column 360 may have operating conditions including a bottoms temperature in the range of about 75° C. (167° F.) to about 150° C. (302° F.) and an overhead pressure in the range of about 75 kPa gauge (11 psig) to about 200 kPa gauge (29 psig). The extractive distillation column 360 may be in downstream communication with the overhead line 44 of the water stripper column 30 and with a bottoms line 46 of the water stripper column.

The recovered methanol is an MTO reactant that can be recycled to the MTO reactor 202, but it is not desirable to recycle the water with the methanol. Hence, the methanol and water rich stream in the net bottoms line 366 may be fed to a methanol stripper column 370 to separate a methanol rich stream in an overhead line 372 from a final water rich stream in a bottoms line 374. The methanol rich stream in the overhead line 372 may then be recycled to the MTO reactor 202 without inert oxygenates that do not react and can otherwise build up in the MTO product unit 310. A portion of the final water rich stream in the bottoms line 374 may be reboiled and recycled to the methanol stripper column 370. The remaining final water rich stream in the net bottoms line 374 may be forwarded along with the water rich recycle stream in line 43 from the water stripper bottoms line 46 to provide the water stream 47. A portion of the water stream 47 may be withdrawn and sent to wastewater treatment in line 51. The remaining water stream in line 53 supplies water to line 19 and 102.

The product olefin stream in the product overhead line 40 carries valuable olefinic products which must be recovered. The concentration section 80 increases the pressure of the product olefin stream necessary for downstream processing such as used in conventional light olefin recovery units. The concentration section 80 may comprise a first knock out drum 82 which separates the product olefin stream into a pressurized first olefin rich stream at a temperature of about 40° C. (104° F.) to about 60° C. (140° F.) and a pressure of about 103 kPa(g) (15 psig) to about 262 kPa(g) (38 psig) in an overhead line 83 and a first aqueous stream rich in oxygenates in a bottoms line 84. The olefin rich stream in the overhead line 83 may be fed to a compressor 85, cooled and directed to a second knockout drum 86. The aqueous stream in the bottoms line 84 is pumped via a manifold line 76 to the return line 32 which returns the water stream with the product water stream in the combined product water stream in line 36 to the water stripper column 30.

The concentration section 80 may comprise a second knock out drum 86 which separates the pressurized first olefin rich stream into a second pressurized olefin rich stream at a pressure of about 103 kPa(g) (15 psig) to about 400 kPa(g) (58 psig), and a temperature of about 27° C. (80° F.) to about 54° C. (130° F.) in an overhead line 87 and a second aqueous stream rich in oxygenates in a bottoms line 88. The second olefin rich stream in the overhead line 87 may be fed to a compressor 89, cooled and directed to a third knockout drum 90. The aqueous stream in the bottoms line 88 is pumped to the return line 32 via the manifold line 76 which returns the water stream with the product water stream in the combined product water stream in line 36 to the water stripper column 30.

The concentration section 80 may comprise a third knock out drum 90 which separates the pressurized second olefin rich stream into a third pressurized olefin rich stream in an overhead line 91 and a third aqueous stream rich in oxygenates in a bottoms line 92. The third olefin rich stream in the overhead line 91 may be fed to the oxygenate absorber column 50. The aqueous stream in the bottoms line 92 is passed to the return line 32 via manifold line 76 which returns the water stream with the product water stream in the combined product water stream in line 36 to the water stripper column 30.

Types of suitable compressors may include centrifugal, positive displacement, piston, diaphragm, screw, and the like. In one embodiment, the compressors 85, 89 in the concentration section 80 are centrifugal compressors. The final discharge pressure can be between about 1 MPa(g) (145 psig) and about 2 MPa(g) (290 psig). The compressor discharge may be cooled to about ambient temperatures using conventional heat transfer methods.

As illustrated in FIG. 2 and according to a preferred embodiment, at least a portion of the compressed product stream via the overhead line 91 is contacted in the oxygenate absorber column 50 at effective conditions to absorb at least a quantity of effluent oxygenates with a cooled lean water stream with no water taken directly from the product separator column 24 without prior removal of oxygenates. In an exemplary embodiment, a solvent stream in line 102 taken from the remaining water stream in line 53 may be passed to the oxygenate absorber column 50. The contacting in the oxygenate absorber column 50 produces an oxygenate lean olefin stream in the overhead line 54 and an oxygenate rich water stream in a bottoms line 52 comprising a quantity of effluent oxygenates. The oxygenate absorber column 50 may have operating conditions including a bottoms temperature range of about 30° C. (86° F.) to about 60° C. (140° F.) and an overhead pressure range of about 700 kPa(g) (101 psig) to about 1 MPa(g) (145 psig). It has been discovered that most of the oxygenates that are produced in the methanol conversion section 111, will be removed from the olefin product. Only carbon monoxide, carbon dioxide, and ethers (DME and DEE) may remain present with the olefin streams.

The oxygenate lean olefin stream in the overhead line 54 may be fed to an absorber separator 60 in which a gaseous olefin stream is taken in an overhead line 61 to a third compressor 62 while water is taken in the bottoms line 59 to the manifold line 76. The gaseous olefin stream in line 61 is compressed in the third compressor 62, and a compressed olefin stream in line 63 is combined with the stream in a stripper overhead line 71, and together partially condensed by cooling in the heat exchanger 64 and fed in line 65 to a stripper separator 66. The stripper separator 66 separates an aqueous stream including oxygenates in the boot in line 67 which feeds the manifold line 76, a light olefin vapor stream in an overhead line 68 comprising propane, propylene, and lighter components and a light olefin liquid stream comprising propane, propylene, and heavier components in line 69. The light olefin liquid stream in line 69 is stripped in a product DME stripper column 70 to remove propane, propylene, and lighter vapors in a stripper overhead stream in line 71 from the heavy olefinic liquid stream in the stripper bottoms line 93. A portion of the heavy olefinic liquid stream in line 93 is reboiled in a reboiler and returned to the DME stripper 70. The heavy olefinic liquid stream in line 93 is withdrawn and can be further processed. Most oxygenates will be stripped into the stripper overhead line 71 and be separated after cooling upon recycle to the stripper separator 66. The stripper separator 66 may operate at a temperature of about 30° C. (86° F.) to about 60° C. (140° F.) and a pressure of about 1.7 MPa(g) (250 psig) to about 2.1 MPa(g) (300 psig).

The light olefin vapor stream in the overhead line 68 is conventionally scrubbed in a caustic scrubber column 302. The crude methanol stream in line 194 may not be purified, so carbon dioxide remaining in the stream in quantities that may require a higher caustic consumption in the caustic scrubber column. So, to avoid the higher caustic consumption and some associated carbon dioxide loss, we propose to remove the bulk of the carbon dioxide in an amine absorber column 302.

The light olefin vapor stream in the overhead line 68 of the stripper separator 66 may be passed through a trayed or packed bulk scrubbing column 302 where it is scrubbed by means of a bulk solvent such as an aqueous solution fed by scrubbing liquid line 304 to remove acid gases including carbon dioxide by extracting them into the aqueous solution. Preferred bulk solvents include Selexol™ available from UOP LLC in Des Plaines, Illinois and amines such as alkanolamines including diethanol amine (DEA), monoethanol amine (MEA), methyl diethanol amine (MDEA), diisopropanol amine (DIPA), and diglycol amine (DGA). Other bulk solvents can be used in place of or in addition to the preferred amines. The lean bulk solvent contacts the light olefin vapor stream in stripper separator overhead line 68 and absorbs acid gas contaminants such as carbon dioxide. The resultant lean light olefin stream is taken out from an overhead outlet of the bulk scrubbing column 302 in a bulk scrubber overhead line 306, and a rich bulk solvent is taken out from the bottoms at a bottom outlet of the bulk scrubbing column 302 in a bulk scrubbing bottoms line 308. The spent bulk solvent from the bottoms may be regenerated and recycled back to the bulk scrubbing column 302 in the scrubbing liquid line 304. The spent bulk scrubbing solvent may be regenerated along with the other solvent that is spent from capturing carbon dioxide from flue gas from fired heaters in the plant or from the MTO regenerator 204. The lean light olefin stream emerges from the bulk scrubber column 302 via the bulk scrubber overhead line 306 and may be fed to the caustic scrubber column 73.

The bulk scrubbing column 302 may be operated with a gas inlet temperature between about 38° C. (100° F.) and about 66° C. (150° F.) and an overhead pressure of about 3 MPa(g) (435 psig) to about 20 MPa(g) (2900 psig). Suitably, the bulk scrubbing column 302 may be operated at a temperature of about 40° C. (104° F.) to about 125° C. (257° F.) and a pressure of about 1200 kPa(g) (175 psig) to about 1600 kPa(g) (230 psig). The temperature of the light olefin vapor stream in the overhead line 68 to the bulk scrubbing column 302 may be between about 20° C. (68° F.) and about 80° C. (176° F.) and the temperature of the bulk solvent stream in the scrubbing liquid line 304 may be between about 20° C. (68° F.) and about 70° C. (158° F.).

The lean light olefin stream in the bulk scrubber overhead line 306 is subsequently scrubbed in a caustic scrubber column 73 by countercurrent contact with a caustic solution in line 42 to absorb remaining acid gases such as carbon dioxide from the vaporous light olefin product stream which exits the caustic scrubber 73 in an overhead line 74. The acid gas rich caustic solution exits caustic scrubber column 73 in line 44 and is sent to wastewater treatment.

The scrubbed light olefinic vapor in overhead line 74 may be refrigerated by propylene refrigerant in a chiller 75 to liquefy part of the light olefin product stream and separated in a drier separator 46 to provide an aqueous stream from a boot in line 347, which is sent to waste water, a vaporous light olefin product stream comprising propylene and lighter components in an overhead line 77 and a liquid light olefin product stream in a bottoms line 78 comprising ethylene and heavier components. The vaporous light olefin stream in the overhead line 77 is dried in a drier 79a to provide a vaporous product olefin stream in line 112. The liquid light olefin stream in the bottoms line 78 is dried in a drier 79b to provide a liquid product olefin stream in line 114. The product olefin streams in lines 112 and 114 are withdrawn and can be further processed.

In accordance with another embodiment of the present disclosure, the integrated process and apparatus for producing light olefins comprises a methanol synthesis unit 101 with a methanol purification section 208 as shown in FIG. 3. Many of the elements in FIG. 3 have the same configuration as in FIG. 1 and bear the same reference number. Elements in FIG. 3 that correspond to elements in FIG. 1 but have a different configuration bear the same reference numeral as in FIG. 1 but are marked with a prime symbol (′).

In accordance with an embodiment, the crude methanol stream in line 196 may be passed to a methanol purification section 208 to separate by-products and/or traces and provide a methanol product stream for the oxygenate conversion unit 200.

In accordance with an exemplary embodiment, the crude methanol stream in line 196 may be passed to the methanol purification section 208 comprising two distillation columns, a first distillation column 210 and a second distillation column 220. Although, in the exemplary embodiment shown in FIG. 3, the methanol purification section 208 comprises two distillation columns, the methanol purification section 208 may comprise more or less than two distillation columns for purifying the crude methanol stream in line 196. The crude methanol stream in line 196 may be passed to the first distillation column 210. In an embodiment, the crude methanol stream in line 196 is heated in heat exchanger 198′ to provide a heated crude methanol stream in line 196′. In the first distillation column 210, the light gas(es) is separated from the crude methanol in a first distillation column overhead stream in line 212. The light gases separated from the crude methanol stream include carbon monoxide, carbon dioxide, methane, hydrogen and dimethyl ether. The first distillation column overhead stream in line 212 is passed to a first overhead receiver 215 after passing through an overhead condenser 211. A condensed first overhead stream in line 213 is passed to the first overhead receiver 215. In the first overhead receiver 215, the light gas(es) is separated in a first overhead receiver vapor stream in line 214. The first overhead receiver vapor stream in line 214 may be passed to a fuel section or used as a fuel perhaps in the combustor 254 in line 256 of FIG. 1. From the first overhead receiver 215, an overhead receiver liquid stream is withdrawn in line 216 and passed to a top of the first distillation column 210.

A first distillation column bottoms stream comprising methanol in line 218 is withdrawn for further separation. The first distillation column bottoms stream comprises methanol comprising less than 100 ppmw of carbon oxide but at least 100 ppmw C₂₊ oxygenates. The first distillation column bottoms stream in line 218 is separated into a first reboiling stream in line 218b and a first distillation column effluent stream in line 218a. The first reboil stream in line 218b is reboiled in a reboiler 219 before passing it to the first distillation column bottom. In accordance with an exemplary embodiment, the first distillation column 210 is operated at a pressure from about 172 kPa(a) (25 psia) to about 1379 kPa(a) (200 psia). In accordance with another exemplary embodiment, the first distillation column is operated at a temperature of about −17° C. (0° F.) to about 177° C. (350° F.).

The first distillation column effluent stream in line 218a includes heavy oxygenates such as C2+ alcohols, ketones, aldehydes that should be removed from the crude methanol stream. Hence the first distillation column effluent stream in line 218a is further separated in a second distillation column 220. In the second distillation column 220, the first distillation column effluent stream in line 218a is separated into a second distillation column overhead stream in line 222 comprising methanol and a second distillation column bottoms stream in line 226. The second distillation column overhead stream comprises methanol with less than 100 ppmw of carbon oxide and less than 100 ppmw C₂₊ oxygenates. The second distillation column overhead is in the vapor phase. Instead of condensing it, a methanol charge stream is taken from the second distillation column overhead line 222 and charged to the MTO reactor 202 in line 199′. A superheater (not shown) may be employed to increase the temperature of the methanol charge stream in line 199′ before charging it to the MTO reactor 202. A portion of the overhead stream in line 222 may be condensed for reflux to the column in line 228. From the heat exchanger 223, a partially condensed second distillation column overhead stream in line 224 is passed to a second overhead receiver 225. In the second overhead receiver 225, the condensed portion of the second distillation column overhead stream in line 224 is recycled to the second distillation column 220.

A second distillation column bottoms stream in line 226 is withdrawn from the column. The second distillation column bottoms stream in line 226 is separated into a second reboiling stream in line 226b and a second distillation column effluent stream in line 226a. The second reboiling stream in line 226b is reboiled in a reboiler 230 before passing it to the second distillation column bottom section. In accordance with an exemplary embodiment, the second distillation column is operated at a pressure from about 3 kPa(a) (5 psia) to about 862 kPa(a) (125 psia). In accordance with yet an exemplary embodiment, the second distillation column operates at a temperature of about 38° C. (100° F.) to about 149° C. (300° F.). The second distillation column effluent stream in line 226a will comprise heavy oxygenates and water, an aqueous oxygenate stream.

In the embodiment of FIG. 1 heavy oxygenates in the crude methanol stream will be processed with the MTO effluent stream in line 207 and be separated into the oxygenate rich stream in line 368 of FIG. 2. In the embodiment of FIG. 3, heavy oxygenates will be concentrated in the aqueous oxygenate stream in the net second distillation bottoms line 226a. It is proposed that the aqueous oxygenate stream in line 226a be transported to line 36 of FIG. 2 to be stripped in the water stripper column 30 where the heavy oxygenates will be stripped from water such as from the product water stream in line 26 to provide an oxygenate stream and also be concentrated into the rich oxygenate stream in line 368.

In accordance with an embodiment of the present disclosure, the methanol purification section 208 may also comprise a third distillation column (not shown) for further removing heavy oxygenates from the crude methanol stream. In accordance with an exemplary embodiment, the third distillation column may operate at a pressure from about 35 kPa (5 psia) to about 345 kPa (50 psia). In accordance with another exemplary embodiment of the present disclosure, the third distillation column may be an atmospheric column operating at about atmospheric pressure. In accordance with yet an exemplary embodiment, the second distillation is operated at a temperature of about 38° C. (100° F.) to about 122° C. (250° F.).

When a third column is also employed, the second distillation column effluent stream in line 226a is separated in the third distillation column to provide an overhead stream comprising methanol and a bottoms stream. The overhead stream may be passed to the MTO reactor 202 along with the methanol product stream from line 222 in MTO charge line 199′.

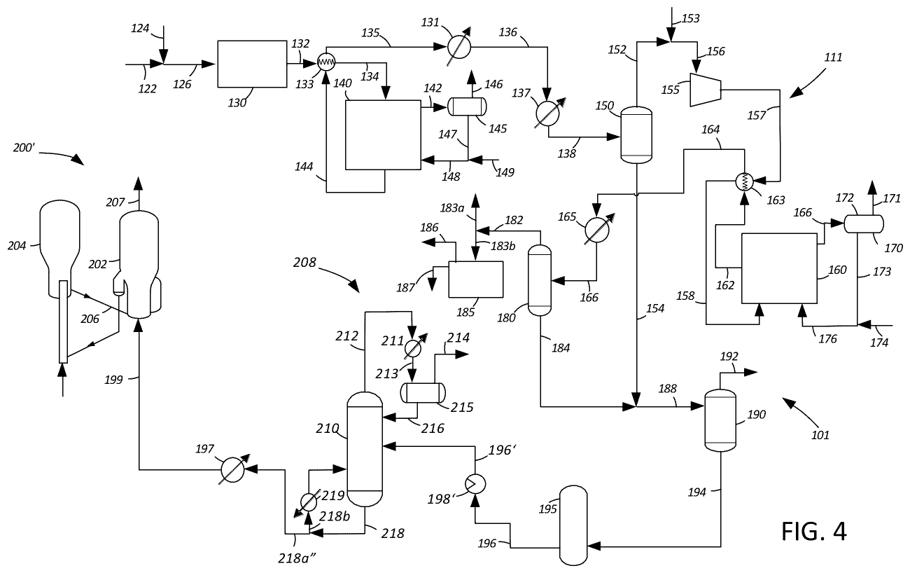

Another exemplary embodiment of the integrated process and apparatus for producing light olefins is shown in FIG. 4. Many of the elements in FIG. 4 have the same configuration as in FIG. 3 and bear the same reference number. Elements in FIG. 4 that correspond to elements in FIG. 3 but have a different configuration bear the same reference numeral as in FIG. 3 but are marked with a double prime symbol (″).

In accordance with an exemplary embodiment, the crude methanol stream in line 196 may be passed to the methanol purification section 208 comprising a single distillation column 210. A bottoms stream comprising methanol in line 218 is withdrawn from the distillation column 210. The single distillation bottoms stream comprising methanol in line 218 comprises less than 100 ppmw of carbon oxide but at least 100 ppmw C₂₊ oxygenates. The bottoms stream in line 218 is separated into a reboiling stream in line 218b and a bottoms effluent stream in line 218a. The reboil stream in line 218b is reboiled in a reboiler 219 before passing it to the distillation column bottom. In accordance with an exemplary embodiment, the distillation column 210 is operated at a pressure from about 172 kPa(a) (25 psia) to about 1379 kPa(a) (200 psia). In accordance with another exemplary embodiment, the distillation column 210 is operated at a temperature of about −17° C. (0° F.) to about 177° C. (350° F.).

The bottoms effluent stream in line 218a″ may include methanol, dimethyl ether, ethanol or combinations thereof. The bottoms effluent stream in line 218a is passed to the oxygenate conversion unit 200. In an embodiment, the bottoms effluent stream in line 218a is preheated and vaporized in a vaporizer 197 to provide a vaporized stream in line 199. The vaporized stream in line 199 is passed to the oxygenate conversion unit 200. The rest of the process is the same as in FIG. 3.

### EXAMPLE

An experimental study was performed to compare the process with a typical MTO process as a base process. Heat duties of the process including the methanol preparation as shown in FIGS. 1 and 3 and the MTO recovery process as shown in FIG. 2 were compared with the similar steps for the base case.

Firstly, the crude methanol feed as shown in FIG. 1 was fed to the MTO recovery process as shown in FIG. 2 for producing the olefins. The methanol was observed to be of 99.85% mass purity for both the base process and the present process. The results of the comparative study for the heat duties are summarized in Table 1.

TABLE 1

| | | Crude methanol |
|---|---|---|
| | | (FIG. 1) and the |
| | | MTO process |
| | Base Process | (FIG. 2) |
| (1) Methanol Purification | 93,401.75 kWh | NA |
| Section Heat Utility Duty | (318.7 mmbtu/hr) | |
| (2) MTO recovery duty | 26,610.85 kWh | 39,799.05 kWh |
| | (90.8 mmbtu/hr) | (135.8 mmbtu/hr) |
| Total Heat Utility Duty | 120,012.60 kWh | 39,799.05 kWh |
| (1 + 2) Required | (409.5 mmbtu/hr) | (135.8 mmbtu/hr) |

| | |
|---|---|
| Heat Utility Duty Savings with Crude | 80,213.55 kWh |
| methanol vs. Base Case= | 273.7 (mmbtu/hr) |

Secondly, the crude methanol was purified using the methanol purification steps as shown in FIG. 3 and thereafter fed to the MTO recovery process as shown in FIG. 2 for producing the olefins. In the methanol purification steps, the feed vaporizer was not required for the present process as compared to the base process. The methanol was observed to be of 99.85% mass purity for both the base process and the present process. The results of the comparative study for the heat duties are summarized in Table 2.

TABLE 2

| | | Methanol purification |
|---|---|---|
| | | (FIG. 3) and the |
| | | MTO process |
| | Base Process | (FIG. 2) |
| (1) Methanol Purification | 93,401.75 kWh | 71,304.19 kWh |
| Section Heat Utility Duty | (318.7 mmbtu/hr) | (243.3 mmbtu/hr) |
| (2) MTO recovery duty | 26,610.85 kWh | −5,685.58 kWh |
| | (90.8 mmbtu/hr) | (−19.4 mmbtu/hr) |
| Total Heat Utility Duty | 120,012.60 kWh | 65,618.61 kWh |
| (1 + 2) Required | (409.5 mmbtu/hr) | (223.9 mmbtu/hr) |

| | |
|---|---|
| Heat Utility Duty Savings with Methanol | 54,393.99 kWh |
| purification vs. Base Case= | (185.6 mmbtu/hr) |

It is evident from the results shown in Tables 1 and 2 that the present process of MTO recovery is more energy efficient than the base process which provides heat utility duty savings. For the crude methanol as feed for the MTO recovery, the present process provided heat utility duty savings of 80,213.55 kWh over the base process. For the methanol purification with the MTO recovery, the present process provided heat utility duty savings of 54,394 kWh over the base process.

### SPECIFIC EMBODIMENTS

While the following is described in conjunction with specific embodiments, it will be understood that this description is intended to illustrate and not limit the scope of the preceding description and the appended claims.

A first embodiment of the invention is a process for producing olefins from carbon oxide comprising providing a crude methanol stream comprising at least 100 ppmw of carbon oxide or at least 100 ppmw C₂₊ oxygenates; and charging the crude methanol stream to an MTO reactor to convert methanol to olefins and produce an MTO effluent stream. An embodiment of the invention is one, any or all of prior embodiments in this paragraph up through the first embodiment in this paragraph further comprising converting carbon oxide to the crude methanol stream. An embodiment of the invention is one, any or all of prior embodiments in this paragraph up through the first embodiment in this paragraph further comprising separating the MTO effluent stream into a heavy oxygenate stream and a product olefin stream. An embodiment of the invention is one, any or all of prior embodiments in this paragraph up through the first embodiment in this paragraph further comprising combusting the heavy oxygenate stream. An embodiment of the invention7 is one, any or all of prior embodiments in this paragraph up through the first embodiment in this paragraph further comprising combusting the heavy oxygenate stream in a CO combustor. An embodiment of the invention is one, any or all of prior embodiments in this paragraph up through the first embodiment in this paragraph further comprising combusting the heavy oxygenate stream in a CO boiler with a flue gas from an MTO regenerator. An embodiment of the invention is one, any or all of prior embodiments in this paragraph up through the first embodiment in this paragraph further comprising absorbing carbon dioxide from the light olefin stream into a bulk solvent to provide a lean light olefin stream. An embodiment of the invention is one, any or all of prior embodiments in this paragraph up through the first embodiment in this paragraph further comprising absorbing carbon dioxide from the lean light olefin stream into a caustic stream to provide a light olefin product stream. An embodiment of the invention is one, any or all of prior embodiments in this paragraph up through the first embodiment in this paragraph further comprising separating aqueous oxygenates from the crude methanol stream to provide an aqueous oxygenate stream before charging the crude methanol stream to the MTO reactor. An embodiment of the invention is one, any or all of prior embodiments in this paragraph up through the first embodiment in this paragraph further comprising quenching the MTO effluent stream to provide a quenched olefin stream; separating the quenched olefin stream to provide a product olefin stream and a product water stream and stripping oxygenates from the product water stream and the aqueous oxygenate stream to provide an oxygenate stream. An embodiment of the invention is one, any or all of prior embodiments in this paragraph up through the first embodiment in this paragraph further comprising separating a heavy oxygenate stream from the oxygenate stream and combusting it.

A second embodiment of the invention is a process for producing olefins from carbon oxide comprising providing a crude methanol stream comprising at least 100 ppmw of carbon oxide or at least 100 ppmw C₂₊ oxygenates; separating light gases from the crude methanol stream to provide an oxygenated methanol stream; separating an aqueous oxygenate stream from the oxygenated methanol stream to provide a methanol stream; and charging the methanol stream to an MTO reactor to convert methanol to olefins and produce an MTO effluent stream. An embodiment of the invention is one, any or all of prior embodiments in this paragraph up through the second embodiment in this paragraph further comprising converting carbon oxide to the crude methanol stream. An embodiment of the invention is one, any or all of prior embodiments in this paragraph up through the second embodiment in this paragraph further comprising separating the aqueous oxygenate stream from the oxygenated methanol stream provides a vaporous methanol stream; and charging the vaporous methanol stream to the MTO reactor. An embodiment of the invention is one, any or all of prior embodiments in this paragraph up through the second embodiment in this paragraph further comprising quenching the MTO effluent stream to provide a quenched olefin stream; separating the quenched olefin stream to provide a product olefin stream and a product water stream and stripping oxygenates from the product water stream and the aqueous oxygenate stream to provide an oxygenate stream. An embodiment of the invention is one, any or all of prior embodiments in this paragraph up through the second embodiment in this paragraph further comprising separating a heavy oxygenate stream from the oxygenate stream and combusting it. An embodiment of the invention is one, any or all of prior embodiments in this paragraph up through the second embodiment in this paragraph further comprising combusting the heavy oxygenate stream in a CO combustor. An embodiment of the invention is one, any or all of prior embodiments in this paragraph up through the second embodiment in this paragraph further comprising combusting the heavy oxygenate stream in a CO boiler with a flue gas from an MTO regenerator.

A third embodiment of the disclosure is a process for producing olefins from carbon oxide comprising providing a crude methanol stream comprising at least 100 ppmw of carbon oxide or at least 100 ppmw C₂₊ oxygenates; separating light gases from the crude methanol stream to provide an oxygenated methanol stream; and charging the methanol stream to an MTO reactor to convert methanol to olefins and produce an MTO effluent stream. An embodiment of the invention is one, any or all of prior embodiments in this paragraph up through the third embodiment in this paragraph further comprising converting carbon oxide to the crude methanol stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for producing olefins from carbon oxide comprising:
providing a crude methanol stream comprising at least 100 ppmw of carbon oxide or at least 100 ppmw C₂₊ oxygenates; and
charging said crude methanol stream to an MTO reactor to convert methanol to olefins and produce an MTO effluent stream.

2. The process of claim 1 further comprising converting carbon oxide to said crude methanol stream.

3. The process of claim 1 further comprising separating said MTO effluent stream into a heavy oxygenate stream and a product olefin stream.

4. The process of claim 3 further comprising combusting said heavy oxygenate stream.

5. The process of claim 4 further comprising combusting said heavy oxygenate stream in a CO combustor.

6. The process of claim 5 further comprising combusting said heavy oxygenate stream in a CO boiler with a flue gas from an MTO regenerator.

7. The process of claim 1 further comprising absorbing carbon dioxide from said light olefin stream into a bulk solvent to provide a lean light olefin stream.

8. The process of claim 7 further comprising absorbing carbon dioxide from said lean light olefin stream into a caustic stream to provide a light olefin product stream.

9. The process of claim 1 further comprising separating aqueous oxygenates from said crude methanol stream to provide an aqueous oxygenate stream before charging said crude methanol stream to said MTO reactor.

10. The process of claim 9 further comprising quenching said MTO effluent stream to provide a quenched olefin stream; separating said quenched olefin stream to provide a product olefin stream and a product water stream and stripping oxygenates from said product water stream and said aqueous oxygenate stream to provide an oxygenate stream.

11. The process of claim 10 further comprising separating a heavy oxygenate stream from said oxygenate stream and combusting it.

12. A process for producing olefins from carbon oxide comprising:
providing a crude methanol stream comprising at least 100 ppmw of carbon oxide or at least 100 ppmw C₂₊ oxygenates;
separating light gases from said crude methanol stream to provide an oxygenated methanol stream;
separating an aqueous oxygenate stream from said oxygenated methanol stream to provide a methanol stream; and
charging said methanol stream to an MTO reactor to convert methanol to olefins and produce an MTO effluent stream.

13. The process of claim 11 further comprising converting carbon oxide to said crude methanol stream.

14. The process of claim 11 further comprising separating said aqueous oxygenate stream from said oxygenated methanol stream provides a vaporous methanol stream; and charging the vaporous methanol stream to said MTO reactor.

15. The process of claim 12 further comprising quenching said MTO effluent stream to provide a quenched olefin stream; separating said quenched olefin stream to provide a product olefin stream and a product water stream and stripping oxygenates from said product water stream and said aqueous oxygenate stream to provide an oxygenate stream.

16. The process of claim 13 further comprising separating a heavy oxygenate stream from said oxygenate stream and combusting it.

17. The process of claim 14 further comprising combusting said heavy oxygenate stream in a CO combustor.

18. The process of claim 4 further comprising combusting said heavy oxygenate stream in a CO boiler with a flue gas from an MTO regenerator.

19. A process for producing olefins from carbon oxide comprising:
providing a crude methanol stream comprising at least 100 ppmw of carbon oxide or at least 100 ppmw C₂₊ oxygenates;
separating light gases from said crude methanol stream to provide an oxygenated methanol stream; and
charging said methanol stream to an MTO reactor to convert methanol to olefins and produce an MTO effluent stream.

20. The process of claim 19 further comprising converting carbon oxide to said crude methanol stream.
